(19) United States
(12) Patent Application Publication
(10) Pub. No.: US 2005/0066987 A1
(43) Pub. Date: Mar. 31, 2005

(54) Nail polish removal tool

(76) Inventor: Daniel Perlman, Arlington, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/790,409

(22) Filed: Mar. 1, 2004

Related U.S. Application Data
(60) Provisional application No. 60/507,423, filed on Sep. 30, 2003.

(51) Int. Cl. A61K 7/04

(52) U.S. Cl. 132/74.5; 401/263

(57) ABSTRACT

A hand-held nail polish removing device that includes a vessel in which a liquid solvent for removing nail polish is stored, in which the vessel includes an opening through which the solvent is added to the device, an applicator element that is chemically resistant and permeable to the solvent, and that is secured within or over the opening in the vessel, in which the largest exposed portion of the applicator element is configured and arranged so that the device can be operated with its major longitudinal axis essentially perpendicular to the surface of a nail while the nail polish is being removed, and a valve assembly that includes a liquid flow valve that can be opened or closed by increasing or decreasing the manual pressure on the solvent applicator element, in which the flow valve is configured and arranged beneath the solvent applicator element.

### CROSS REFERENCE TO RELATED APPLICATIONS

This application claims the priority of provisional patent application No. 60/507,423, filed Sep. 30, 2003 and incorporates the same by reference.

### STATEMENT REGARDING FEDERALLY SPONSORED RESEARCH OR DEVELOPMENT

N/A

### BACKGROUND OF THE INVENTION

The present invention relates to nail care cosmetics products, and more particularly to the field of devices for storing and applying liquid nail polish removers, and a method for facilitating removal of lacquers from fingernails and toenails.

None of the references cited below are admitted to be prior art to the present invention.

For reasons of economy and efficacy, the most common solvent systems used to remove nail polishes (also known as lacquers) have been acetone and ethyl acetate. However, many problems including skin irritation, and skin and cuticle desiccation are associated with the use of these solvents, and many patents have been issued describing remedies for these problems [see Curtis (U.S. Pat. No. 4,485,037), Hofmann (U.S. Pat. No. 4,824,662), Hofmann (U.S. Pat. No. 5,077,038), Helioff et al. (U.S. Pat. No. 5,024,779), Remz et al. (U.S. Pat. No. 5,294,435), Miner et al. (U.S. Pat. No. 5,342,536) and Faryniarz et al. (U.S. Pat. No. 5,486,305)].

Solvent systems other than acetone and ethyl acetate have been described in the patent literature for nail polish lacquer removers, but have met with limited commercial success. These alternative systems have distinct drawbacks including health risks and environmental toxicity problems, [see Adams (U.S. Pat. No. 4,543,206), Dotolo et al. (U.S. Pat. No. 5,346,652), and Bayless (U.S. Pat. No. 5,372,742)].

Several patents have focussed on thickening or gelling systems for enhancing nail polish lacquer removers [see Minton et al. (U.S. Pat. No. 4,197,212), Day (U.S. Pat. No. 4,804,486), Miner (U.S. Pat. No. 5,543,085)].

While acetone and methyl acetate (but not ethyl acetate) are currently EPA-unlisted solvents, they are undesirable for many reasons including their unpleasant smell, their excessive volatility and rapid drying (necessitating application of relatively large quantities of solvent), their vapor being irritating to the eyes, and their desiccation and irritation of the cuticle and skin around the nail. Acetone is also known to cause bronchial irritation, and skin erythema via topical exposure. Consequently, Applicant has described in U.S. Pat. No. 6,521,572 the use of an essentially odor-free liquid nail polish remover having a substantially reduced volatility, based upon the organic solvent, gamma butyrolactone This solvent allows a very small volume (a few microliters) of the remover liquid to be applied by brush and to persist long enough to soften and dissolve the lacquer on a fingernail. The disclosure of the '572 patent is incorporated by reference herein.

Pen-like devices are known for dispensing small amounts of a solvent for nail polish removal. These pens have nib-like tips designed to dispense small amounts of a solvent over a portion of the fingernail to remove an area of excess or damaged lacquer. None of these devices are well adapted for rapid stripping of the entire lacquer coating from one or more fingernails. Marker nibs are rigid or semi-rigid extensions of the pen that function to deposit an ink or paint. Nibs are composed of a felt, synthetic fiber or microporous thermoplastic material. When nail polish remover solvent rather than ink or paint is dispensed from a conventional marker, the solvent must migrate through the nib structure that includes a nib stem extending outward from the body of the pen. Control of solvent flow to the nail surface through the nib is often a problem regardless of whether a flow valve is present in the pen at the proximal or inner end of the nib. For example, if no flow valve is present (e.g., in a simple felt-tipped pen), the rate of solvent flow tends to be insufficient for convenient removal of nail polish. On the other hand, if a valve is present it is not unusual to experience a delayed flood of solvent onto the nail because there is a lag between the time the nib is pressed and the solvent arriving at the distal or outer end of the nib. Moreover, the nib is typically much smaller than a fingernail so that considerable back and forth motion is required for the nib to coat the nail's surface with solvent to softened the lacquer for removal from the nail.

A number of other hand-held applicator devices are described in the patent literature for storing, dispensing and/or applying liquid nail polish removers to nails. For example, Jones (U.S. Pat. No. 3,592,202) describes a combined doubled-ended liquid nail polish applicator and liquid remover device that includes flexible storage portions that may be compressed to eject either liquid over separate applicator brushes. D'Agostino (U.S. Pat. No. 5,439,012) describes a hand-held clamping and holding device for cotton or other material allowing the material to be saturated with a solvent and applied to a lacquered nail surface without the need for hand contact with the solvent. Bourassa (U.S. Pat. No. 6,148,828) describes an elongated cosmetic dispensing device having a wiping tip and slidable cleaning ribbon saturated with a cleaning solution such as a nail polish remover. Harrison, et al. (U.S. Pat. No. 6,209,548) describes a pen-like applicator for nail polish paints that includes a barrel for storing the nail paint and a protruding chisel-shaped nib for applying the paint. The barrel may have a flow-restricting valve, and the paint is a low viscosity water-based paint to permit the paint to flow through the nib. A very similar pen-like device commercially marketed under the name “Nail Dabber” contains a nail polish removing solvent described by Baker (U.S. Pat. No. 6,225,269). The device has a barrel, a push-valve that can open and close, and a hard chisel-shaped nib whose porous stem extends into the barrel to open the valve and allow capillary flow of solvent to the nail.

Marker pens are known for dispensing ink from a rigid or semi-rigid writing nib which typically is a felt-tipped nib, a porous fiber nib or porous polyethylene nib. Bingo markers are also known for imprinting an ink stamp on bingo sheets. These bingo markers typically include a 1-2 cm diameter flat or dome-shaped ink applicator pad of varying thickness (whose outer surface is either sculpted or not for imprinting decorative designs) fabricated from a spongy synthetic foam material. When the marker is pushed down on a bingo card, ink is released from a valved end of the marker into the foam applicator pad, which thereafter leaves a stamped imprint on the sheets.

### BRIEF SUMMARY OF THE INVENTION

This invention concerns nail polish removal from fingernails and toenails (collectively termed nails), and, more specifically, to providing a hand-held liquid solvent-dispensing and application device. The device includes a vessel similar in size to a marker or pen, that includes both a liquid shut-off valve assembly (e.g., a mechanical push-valve) and a solvent applicator element (e.g., a compliant layer of solvent-permeable material) that is positioned outside the valve. The applicator element allows distribution of a nail polish remover solvent over the surface of the nail and scrubbing of the nail surface to loosen and remove the nail polish. The material used in the applicator element is preferably a tufted or textured fabric which is of a material such as Dacron which is chemically resistant to the remover solvent and permeable to the solvent. The applicator element can also be an open cell mesh or foam material, or other porous material, provided that it is also resistant to the remover solvent and compliant to the nail surface. The invention also includes a method for applying the remover solvent to the nail surface. This method differs from that previously used in which a pen fitted with a porous marker-style nib is provided for removing or repairing small areas of lacquer on a nail. Furthermore, with the presently described dispensing device, there is almost no delay between the time the compliant applicator is pressed downward against the nail surface, and the time the liquid shut-off valve (i.e., push valve) releases solvent through the applicator material onto the nail. The compliant applicator element can be easily configured and amply sized to cover an entire fingernail with one or two back and forth passes over the nail.

The present invention is intended to provide improved economy of solvent use, and improved convenience when compared with the traditional and rather messy cotton ball that is saturated with solvent for application to the nail. It is also intended to overcome the previously described limitations inherent in nib-containing nail polish correction pens. The present invention utilizes a suitably sized applicator element that includes a layer of compliant material that is both solvent-resistant and solvent-permeable and that can transmit but not retain appreciable quantities of liquid solvent. The applicator portion is also enlarged relative to the size of a writing nib and forms one end of the marker. It is located distal or outside the valve assembly that controls the flow of solvent. Rather than being pointed or protruding like a chisel tip or bullet tip, the surface of the applicator is typically flat or somewhat rounded (preferably convex outward) to facilitate contact and smooth movement over the surface of the nail. The applicator is adequately sized (preferably between 0.5 cm and 2 cm in diameter or width) so that it can cover between 25% and 100% of the nail at a time. In that manner, an individual nail can be covered with solvent in a few seconds. In order to allow time to remove solvent-softened nail lacquer using a tissue, cotton material or paper towel, for example, it is desirable that the solvent not evaporate too rapidly. Such solvents having reduced volatility compared to acetone or methyl and ethyl acetate are described by Perlman (U.S. Pat. No. 6,521,572).

The present invention is critically different in form and function from earlier nail polish remover pens by being free of the traditional nib. By the term “nib” is meant a porous structure that extends outward from the barrel or body of a marker, and that is suitably sized, shaped, and hard enough for writing, and that limits the flow of liquids such as inks and solvents by its internal structure (e.g., a fiber channel structure or a tortuous path pore structure that allows only capillary flow).

The applicator in accordance with the invention is softer, larger and more compliant than known nib devices so that it may contact a much greater portion of the nail surface at a given time. In addition, because the applicator surface is generally aligned perpendicular to the axis of the device rather than parallel (as in the case of the nib), the applicator can be more easily manipulated and controlled than a conventional nib, thereby adding utility to the device. Besides allowing convenient and rapid coating of the nail with lacquer removal solvent, the applicator can be fabricated from a textured material that helps to scrub, disrupt and remove nail lacquer from the nail as the lacquer becomes softened by the solvent. Thus, the applicator plays a dual role of both applying solvent and then dislodging lacquer as it is softened. Moreover, since the applicator material does not retain appreciable volumes of lacquer residue, the applicator is easily cleanable by wiping off the residue with an absorbent tissue or other material.

### Definitions

As used in this description and the accompanying claims, the following terms shall have the meanings indicated, unless the context otherwise requires:

The term “vessel” as used herein, is a hollow and generally elongated container or reservoir that is chemically resistant to the solvent or blend of solvents used in the device. The vessel is fabricated from a thermoplastic resin that will allow this reservoir to remain impermeable and unreactive with the nail polish remover solvent composition over an extended period of time, e.g., for at least a year.

The term “liquid solvent” as used herein, refers to a nail polish-removing (dissolving) solvent that is a liquid (or, optionally, thickened liquid) at room temperature and under normal conditions of use. The solvent is based upon either a single organic solvent or a blend of such solvents that have the ability to dissolve dried nail lacquers including those formed from either aqueous-based nail polishes or organic solvent-based nail polishes. The solvent may include one or more of the following: surfactants, emollients and/or humectants, preservatives, antioxidants, stabilizers, vitamins, antioxidants, colors, fragrances, denaturants and diluents. For example, polysorbate 20 is a useful surfactant at level of approximately 0.1%-2%. Glycerol (0.1%-2%) is a useful emollient, propylene glycol or dipropylene glycol (0.2%-2%) is a useful stabilizer, TBHQ may be a useful antioxidant, denatonium benzoate is a useful bittering agent and denaturant to prevent accidental ingestion, and water or dipropylene glycol may be a useful diluent.

The term “applicator element” is meant to include a wide range of materials that can be die-cut, punched, molded, extruded or otherwise formed into a useful geometry for applying solvent to a nail, provided that the element material is chemically resistant and permeable to the nail polish-removing solvent. This element may be secured, i.e., attached, almost anywhere inside or outside the opening in the vessel as long as at least a portion of the element is oriented outward and exposed so as to contact the nail with the solvent that is being transmitted through the element when the flow valve in device is opened. By the term “chemically resistant” is meant that the applicator material (and any other parts of the device that are exposed to solvent) are not structurally damaged, i.e., not substantially weakened or dissolved, during the lifetime of the device, e.g., 1-2 years. By the term “permeable” is meant that the solvent can readily pass through the material, e.g., within a matter of seconds, to be available for coating the nail.

The term “largest exposed portion” refers to the applicator element and the solvent-permeable material in the applicator that is exposed, i.e., faces the outside air, around the device. In the embodiment of the present invention shown in FIG. 1, the largest exposed portion is labeled 40, and is a dome-shaped surface that can be used to apply solvent to the nail.

The expression “can be operated with its major longitudinal axis essentially perpendicular to the surface of a nail” means that the device can be conveniently held at approximately a right angle to the main surface portion of the fingernail or toenail while releasing solvent onto the nail and rubbing the applicator surface over this surface to remove nail lacquer.

The term “liquid flow valve” refers to any useful and reversible closure mechanism that can control the flow of the solvent out of the device, e.g., by opening and closing, as manual pressure is either applied or released from the applicator element.

The phrase “geometric shape of the largest exposed portion of the applicator element” means that the principal exposed surface of the applicator that contacts the fingernail can be formed into any of a variety of geometric shapes to reach the various surfaces of the nail during lacquer removal. For example, a flat disc-shaped applicator may be preferred by some people, while others may prefer to use an applicator with any of a variety of contoured surfaces, e.g., convex outward (dome-shaped), concave outward (hollowed out end) and polyhedral (multi-planar) shapes.

The material for fabricating the applicator can be almost any durable material that is permeable and is not degraded by the solvent, including open cell foams, fabric materials, non-woven fiber materials, aligned fiber materials, porous plastic materials and combinations thereof. Many thermoplastic resins provide options for fabricating the applicator including polyolefins, polyesters, polyurethanes, polycarbonates, nylons and combinations thereof.

The characterization of the applicator element as “compliant to the nail surface during use” means that when the applicator material is pressed against the nail, it has sufficient elasticity or “give” to assume the local shape of the nail and/or cuticle surface. This is a useful property since it allows the applicator surface to contact, and thereby apply the nail polish-remover solvent to recessed and/or irregular portions of the nail that are coated with nail lacquer.

The term “push-valve” is known in the art, and refers to a type of liquid “shut-off” valve that opens or closes by applying or releasing pressure from the valve. In the present invention, this pressure is applied to the valve via the applicator element that lies outside the valve and contacts the nail.

The fact that the use of a traditional nib is obviated by the use of a compliant applicator material that transports solvent to the nail is an important element of the present invention, and is discussed elsewhere in the text.

In a first aspect, the present invention features a hand-held nail polish removing device that includes: (i) a vessel in which a liquid solvent for removing nail polish is stored, in which the vessel has an opening through which the solvent is added to the device, (ii) an applicator element that is chemically resistant and permeable to the solvent, and that is secured within or over the opening in the vessel, where the largest exposed portion of the applicator element is configured and arranged so that the device can be operated with its major longitudinal axis essentially perpendicular to the surface of a nail while the nail polish is being removed, and (iii) a valve assembly that includes a liquid flow valve that can be opened or closed by increasing or decreasing the manual pressure on the applicator element, in which the flow valve is configured and arranged beneath the solvent applicator element.

In preferred embodiments, the vessel portion of the device is configured and sized to be conveniently hand held. The vessel has a generally elongated shape with any of a variety of cross-sectional geometries selected from the group consisting of round, oval, polygon and miscellaneous contoured shapes. These contoured shapes may include concave inward sides for gripping the device. The vessel is preferably sized to hold between 10 ml and 100 ml of nail polish removing solvent. If approximately 50 microliters of solvent is used to remove lacquer from each nail, 0.5 ml will be used to clean ten nails, and a vessel having a 20 ml capacity will allow 40 solvent cleanings, ten fingernails per cleaning.

In another preferred embodiment, the device's applicator element used for applying solvent to the nail has a surface area of between 0.2 and 4.0 square centimeters. More preferably, the solvent applicator's surface area is between 0.5 and 2.0 square centimeters. These areas are of the same order of magnitude as the surface area of the fingernail so that only a few back and forth motions with the applicator are sufficient to cover the nail.

In another preferred embodiment, the overall geometric shape of the solvent applicator's surface is selected from the group consisting of flat, convex outward, concave outward and polyhedral shapes. These different shapes may provide varying degrees of access to different portions of nails and cuticles.

The solvent applicator element is fabricated of a material which preferably is tufted or textured and which is chemically resistant to the nail polish remover solvent and porous to the solvent. The material also has a low capacity for retaining an appreciable volume of solvent and a low capacity for retaining residue of the removed lacquer. The material is also preferably slightly abrasive. The solvent applicator element can also be fabricated of other materials such as open cell mesh or foams, fabric materials, non-woven fiber materials, aligned fiber materials, porous plastic materials and combinations thereof. These various physical forms of the applicator material are fabricated from a polymeric resin material selected from the group consisting of polyolefins, polyesters, polyurethanes, polycarbonates, nylons and combinations thereof.

In a preferred embodiment, the applicator material is, furthermore, compliant to the nail surface during use so as to increase the extent of contact between the applicator's surface and the surface of the nail. This increased contact facilitates coverage of the nail with the lacquer removing solvent.

In another preferred embodiment, the valve assembly includes a push-valve that can be opened by manual pressure applied to the solvent applicator element. Accordingly, pressing the device down against the surface of the nail opens the push valve, and releasing this pressure allows the valve to re-close.

In yet another preferred embodiment, the structure, configuration and arrangement of the applicator element and the flow valve obviate the need for a nib to transport the solvent from the vessel to the nail.

In another aspect, the present invention features a method of applying a nail polish remover solvent to a lacquer-coated nail. The method includes the steps of: (i) providing the nail polish removing device described above, then contacting the surface of the lacquer-coated nail with the solvent applicator element of the device, (ii) applying downward manual pressure on the lacquer-coated nail with the applicator element for a period of time sufficient to release an adequate quantity of the solvent, (iii) moving the applicator element over the surface of the lacquer-coated nail until the solvent has covered the lacquer-coated nail, and (iv) optionally scrubbing the surface of the nail to loosen and remove the softened nail polish.

In another aspect of this method, the above applying, moving and scrubbing steps can be carried out at the same time.

In preferred embodiments of the above two aspects of the method, as an option, it may be useful to wait for a period of time, for example up to about 30 seconds, after applying the solvent for the lacquer on the nail to be softened. Then, the solvent applicator element can be rubbed back and forth over the surface of the nail until the lacquer has been displaced, i.e., removed from the nail.

### BRIEF DESCRIPTION OF THE SEVERAL VIEWS OF THE DRAWINGS

FIG. 1 is an exploded perspective view in partial section showing a pen-like dispensing device for nail polish remover that includes a solvent reservoir, a push valve assembly (with applicator for solvent) and a protective cap; and

FIG. 2 is an enlarged elevation view in partial section of an applicator element having tufts on a fabric backing.

### DETAILED DESCRIPTION OF THE INVENTION

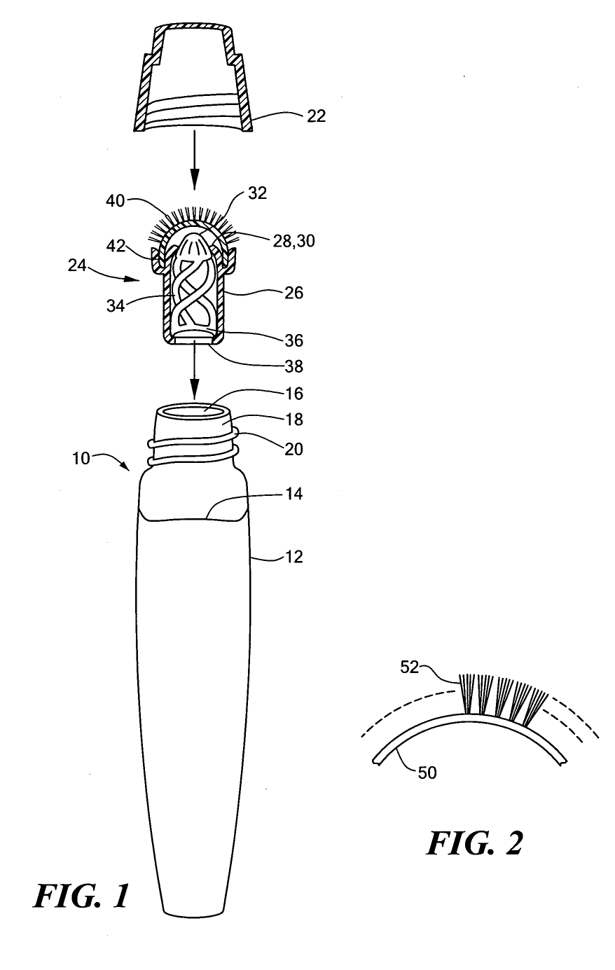

Referring to FIG. 1, dispensing device 10 includes a vessel or reservoir 12 that is sized to hold between 10 ml and 100 ml (preferably approximately 20 ml) of nail polish remover solvent. This portion of the device is fabricated by blow molding from high density polyethylene. Depending upon the aggressiveness of the chemical solvent, a variety of alternative solvent-resistant polymers may be considered such as polypropylene, polyester or nylon. Nail polish remover solvent 14 is loaded into the device 10 through a cylindrically-shaped opening 16 formed in the neck portion 18 of reservoir 12. An external thread 20 formed on the outside of the neck portion 18 allows an internally threaded cap 22 to be removably attached to the neck portion 18 to reduce evaporation of any nail polish remover solvent. Push valve assembly 24 includes a hollow and generally cylindrically shaped tube portion 26 that is sized to form a liquid-tight seal and frictional fit inside opening 16. Tube portion 26, in turn, includes a reduced diameter round upper opening 28 defined and bordered by sealing rim 30 that cooperates and mates with a rounded conical sealing plug 32 that is co-axial with, and inside tube portion 26, and forms a liquid-tight seal with sealing rim 30. Sealing plug 32 is integrally fabricated and mounted upon, and pressed upward by a triple helical plastic spring 34 whose three helices are united by a circular hoop 36 that is sized so as to be captively held within, and just above the reduced diameter lower opening 38 of tube portion 26. Applicator element or “applicator” 40 is typically fabricated from a compliant yet durable textured material, e.g., a tufted woven fabric, that is permeable, absorbent and resistant to the solvent, and that allows a selected solvent or solvent blend to be applied to the lacquer nail polish coating on the surface of a nail.

Applicator 40 is generally positioned immediately above, and may be attached to, push-valve assembly 24 that includes conical sealing plug 32. For example, the material that is used to fabricate applicator 40 can be held in place above, and just outside, upper opening 28 (and just above sealing plug 32) using a hooking or crimping seal 42 for securing the material. In use, when pressed against a nail's surface with a force exceeding the resistive force of spring 34 (the force needed to begin compressing spring 34), applicator 40 causes sealing plug 32 to be pushed inward and release some of the nail polish remover solvent 14. The ability of applicator 40 to comply to the nail, i.e., its ability to flex and/or compress, helps promote rapid and uniform application of the solvent to the entire nail surface. The applicator 40 is preferably composed of a fabric or other material that includes tufted or otherwise mildly abrasive or scouring elements that protrude from the surface of a backing material to help displace the nail polish as it softens and dissolves upon exposure to the solvent. Such an applicator is illustrated in FIG. 2 and includes a fabric backing 50 and tufts 52 which outwardly extend from the backing to provide a tufted or rug-like textured surface. In one embodiment the tufts are about 1/8 inch long. The backing is a thin woven material having an open structure that allows transmission of the solvent. A paper tissue may be used in conjunction with device 10 and its solvent 14 to remove residues of softened and dissolved nail polish from both the nails and from the surface of the applicator. If any residues of nail polish remain on the nail after one application of solvent 14, the application process can be repeated.

Low vapor pressure organic solvents (abbreviated “LVP-VOC”) as defined by the California Air Resources Board (abbreviated “CARB”) are currently preferred for use in the present invention. Certain LVP-VOCs including propylene carbonate and dibasic esters such as dimethyl adipate have been tested by Applicant and found to be particularly effective in the presently invented dispensing device. In addition, a small number of volatile organic compound (abbreviated “VOC”) solvents and low-reactive organic compounds (LR-VOC) have been exempted by CARB from the list of excluded solvents, and therefore may be utilized in nail polish removers used in conjunction with the present invention. The LR-VOC exempted solvents including acetone and methyl acetate may be considered as alternatives or diluent additives to LVP-VOC solvents used in this invention. For a definition of terms including LVP-VOC, and a complete listing of exempted VOCs and LR-VOCs, which can be considered for use in the presently invented device, see “Regulation for Reducing Volatile Organic Compound Emissions From Consumer Products;” Final Regulation Order, on the Internet at www.arb.ca.gov/regact/midterm2/finreg.pdf>. This document is included herein by reference in its entirety.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The specific methods and compositions described herein as presently representative of preferred embodiments are exemplary and are not intended as limitations on the scope of the invention. Changes therein and other uses will occur to those skilled in the art which are encompassed within the spirit of the invention are defined by the scope of the claims.

It will be readily apparent to one skilled in the art that varying substitutions and modifications may be made to the invention disclosed herein without departing from the scope and spirit of the invention. For example, those skilled in the art will recognize that the invention may suitably be practiced using any of a variety of materials to form the structural parts of the device provided that they are chemically and physically stable, i.e., unreactive and insoluble, when contacted by a particular nail polish remover solvent.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is not intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group. For example, if there are alternatives A, B, and C, all of the following possibilities are included: A separately, B separately, C separately, A and B, A and C, B and C, and A and B and C. Thus, the embodiments expressly include any subset or subgroup of those alternatives, for example, any subset of the types of resins used in the applicator material. While each such subset or subgroup could be listed separately, for the sake of brevity, such a listing is replaced by the present description.

While certain embodiments and examples have been used to describe the present invention, many variations are possible and are within the spirit and scope of the invention. Such variations will be apparent to those skilled in the art upon inspection of the specification and claims herein.

Other embodiments are within the following claims.

1. A hand-held nail polish removing device comprising:
a vessel in which a liquid solvent for removing nail polish is stored, said vessel having an opening through which said solvent is added to said device;
an applicator element that is chemically resistant and permeable to said solvent, and that can transmit but not retain the solvent;
wherein said applicator element is secured over said opening in said vessel, wherein the largest exposed portion of said applicator element is configured and arranged so that said device can be operated with its major longitudinal axis essentially perpendicular to the surface of a nail while said nail polish is being removed; and
a valve assembly comprising a liquid flow valve that can be opened or closed by increasing or decreasing the manual pressure on said applicator element, wherein said flow valve is configured and arranged beneath said applicator element.

2. The device of claim 1 wherein said vessel is configured and sized to be hand-held, wherein said vessel has a generally elongated shape with a cross-sectional geometry selected from the group consisting of round, oval, polygon and contoured shapes.

3. The device of claim 1 wherein said vessel is sized to hold between 10 ml and 100 ml.

4. The device of claim 1 wherein said largest exposed portion of said applicator element has an area of between 0.2 and 4.0 square centimeters.

5. The device of claim 4 wherein said largest exposed portion of said applicator element has an area of between 0.5 and 2.0 square centimeters.

6. The device of claim 1 wherein the geometric shape of said largest exposed portion of said applicator element is selected from the group consisting of flat, convex outward, concave outward and polyhedral shapes.

7. The device of claim 1 wherein said applicator element comprises a permeable material selected from the group consisting of open cell foams, fabric materials, non-woven fiber materials, aligned fiber materials, porous plastic materials and combinations thereof.

8. The device of claim 7 wherein said permeable material is fabricated from a resin selected from the group consisting of polyolefins, polyesters, polyurethanes, polycarbonates, nylons and combinations thereof.

9. The device of claim 1 wherein said largest exposed portion of said applicator element is compliant to the nail surface during use.

10. The device of claim 1 wherein said valve assembly comprises a push-valve that can be opened by manual pressure applied to said applicator element.

11. The device of claim 1, wherein the structure, configuration and arrangement of said applicator element and said flow valve obviate the need for a nib to transport said solvent from said vessel to said nail.

12. A method of applying a nail polish remover solvent to a lacquer-coated nail, using a hand held device which holds a quantity of liquid solvent and having a valved end for release of solvent during downward manual pressure on the valved end, comprising the steps of:
providing an applicator element on the valved end of the hand held device that is chemically resistant and permeable to the solvent and that can transmit but not retain the solvent;
contacting the surface of said lacquer-coated nail with said applicator element;
applying downward manual pressure on said lacquer-coated nail with said applicator element for a period of time sufficient to release an adequate quantity of said solvent;
moving said applicator element over the surface of said lacquer-coated nail until said adequate quantity of said solvent has covered said lacquer-coated nail; and
optionally scrubbing said applicator element over the surface of said lacquer-coated nail to loosen and remove the lacquer.

13. The method of claim 12 wherein said applying, moving and scrubbing steps are carried out simultaneously.

14. The method of claim 12 further comprising:
waiting until said adequate quantity of said solvent has softened the lacquer on the nail; and
rubbing said applicator element over the surface of said nail until said lacquer has been displaced from said nail.

15. The method of claim 13 further comprising:
waiting until said adequate quantity of said solvent has softened the lacquer on the nail; and
scrubbing said applicator element over the surface of said nail until said lacquer has been displaced from said nail.

16. The method of claim 12 further comprising:
removing the residue of dissolved lacquer from the applicator element.

17. The method of claim 16 wherein the step of removing the residue includes:
removing with an absorbent material the residue of dissolved lacquer from the applicator element.
